(19) United States
(12) Patent Application Publication
(10) Pub. No.: US 2023/0383323 A1
(43) Pub. Date: Nov. 30, 2023

(54) BACTERIAL STRAIN FOR FERMENTING WHEAT BRAN TO SYNTHESIZE EXTRACELLULAR POLYSACCHARIDE

(71) Applicant: BRIGHT DAIRY & FOOD CO., LTD, Shanghai (CN)

(72) Inventors: Jin HAN, Shanghai (CN); Zhenmin LIU, Shanghai (CN); Zhengjun WU, Shanghai (CN); Chengxiao GUO, Shanghai (CN); Chunping YOU, Shanghai (CN)

(73) Assignee: BRIGHT DAIRY & FOOD CO., LTD, Shanghai (CN)

(21) Appl. No.: 17/926,634

(22) PCT Filed: Sep. 26, 2021
(86) PCT No.: PCT/CN2021/120703

§ 371 Date: Nov. 21, 2022

(30) Foreign Application Priority Data
Jul. 23, 2021 (CN) 202110837446.X

(51) Int. Cl. C12P 19/04

(52) U.S. Cl. CPC C12P 19/04

(57) ABSTRACT

A strain of Paenibacillus sp. for fermenting wheat bran to synthesize an extracellular polysaccharide is disclosed. A weight-average molecular weight of the extracellular polysaccharide synthesized by the strain is 300,800 to 451,200 daltons, and the extracellular polysaccharide is an acidic heteropolysaccharide composed of glucuronic acid, glucose and fucose in a molar ratio of (1.55 to 1.60):1:(1.63 to 1.72); a backbone of the extracellular polysaccharide is composed of a 1,3-linked glucose residue, a 1,3-linked fucose residue, a 1,3,4-linked fucose residue and a 1,4-linked glucuronic acid residue, a branch point is located at a 0-4 position of the 1,3,4-linked fucose residue, and a branch chain is composed of terminally linked glucuronic acid residues, and the extracellular polysaccharide is composed of a repeating unit shown in Formula I. The extracellular polysaccharide has a certain immunomodulatory effect, and has a good application prospect in food, medicine and related fields.

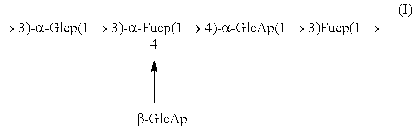

### CROSS REFERENCE TO THE RELATED APPLICATIONS

This application is the national phase entry of International Application No. PCT/CN2021/120703, filed on Sep. 26, 2021, which is based upon and claims priority to Chinese Patent Application No. 202110837446.X, filed on Jul. 23, 2021, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBDZ044 Sequence Listing.txt, created on 11/17/2022, and is 2,243 bytes in size.

### TECHNICAL FIELD

The present invention belongs to the field of microorganisms, and in particular, relates to a bacterial strain for fermenting wheat bran to synthesize an extracellular polysaccharide.

### BACKGROUND

Since the 1960s, polysaccharides have been considered as a broad-spectrum non-specific immunostimulant, which can enhance the functions of cellular immunity and humoral immunity of host cells, such as activating macrophages, T cells, B cells, NK cells, etc., and activating complement and induction to generate interferon, etc., whose effects will be beneficial to in enhancing the human body's non-specific defense function in anti-virus, anti-tumor, anti-radiation and other aspects.

According to their sources, the polysaccharides can be divided into animal polysaccharides, plant polysaccharides and microbial polysaccharides, wherein the microbial polysaccharides are synthesized by microorganisms metabolizing carbohydrates. Usually, the cultivation medium for microbial polysaccharide synthesis is a defined or semi-defined one, such as MRS, TYC, etc. However, relatively little research has been done on the synthesis of the microbial polysaccharides by using natural raw materials, especially agriculturally processed by-products (such as wheat bran). In addition, research on bacterial strains that can ferment bran to synthesize an extracellular polysaccharide is also relatively scarce.

Therefore, it is necessary to develop a strain using the wheat bran as a fermentation medium. On the one hand, the added value of agricultural by-products is improved, and the preparation cost of microbial extracellular polysaccharides is reduced. On the other hand, new extracellular polysaccharides can be obtained to meet the needs of research, and meanwhile, the types of microbial extracellular polysaccharides with excellent performance and wide application are also enriched.

### SUMMARY

The present invention aims to provide a bacterial strain for fermenting wheat bran to synthesize an extracellular polysaccharide. The extracellular polysaccharide synthesized by the disclosed bacterial strain in the present invention has various biological effects, and has a good application prospect in food, medicine and related fields. The present invention solves the above technical problems mainly through the following technical solutions.

The present invention provides a strain of Paenibacillus sp. for fermenting wheat bran to synthesize an extracellular polysaccharide, and a preservation number of the strain is CGMCC NO.8333. The strain was preserved in the China General Microbiological Culture Collection Center (CGMCC) on Oct. 14, 2013. The preservation address is: No. 3 Courtyard 1 West Beichen Road, Chaoyang District, Beijing 100101, China.

Preferably, the strain can ferment the wheat bran to synthesize an extracellular polysaccharide.

Preferably, a weight-average molecular weight of the extracellular polysaccharide synthesized by the strain is 300,800 to 451,200 daltons.

Preferably, the extracellular polysaccharide synthesized by the strain is an acidic heteropolysaccharide composed of glucuronic acid, glucose and fucose in a molar ratio of (1.55 to 1.60): 1:(1.63 to 1.72).

Preferably, a backbone of the extracellular polysaccharide synthesized by the strain is composed of a 1,3-linked glucose residue, a 1,3-linked fucose residue, a 1,3,4-linked fucose residue and a 1,4-linked glucuronic acid residue, a branch point is located at a 0-4 position of the 1,3,4-linked fucose residue, and a branch chain is composed of terminally linked glucuronic acid residues.

Preferably, the extracellular polysaccharide synthesized by the strain is composed of a

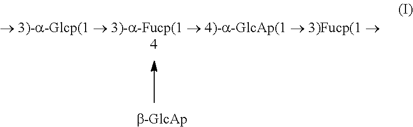

repeating unit shown in Formula I.

The present invention further provides use of an extracellular polysaccharide synthesized by a strain of Paenibacillus sp. in fields of food and medicine.

The present invention further provides use of an extracellular polysaccharide synthesized by a strain of Paenibacillus sp. in preparation of immunomodulatory medicines.

Preferably, a concentration of the extracellular polysaccharide synthesized by the strain lower than 100 μg/mL exhibits obvious immunomodulatory activity.

Compared with the prior art, the present invention has the following positive progressive effects.

The technical solution of the present invention discloses a strain of Paenibacillus sp. that can ferment wheat bran to synthesize an extracellular polysaccharide for the first time. The extracellular polysaccharide synthesized by the strain has a clear and special structure, not only is safe, but also has various biological activities, such as enhancing the phagocytic function of macrophages and promoting the release of cytokines from the macrophages, so that it has significant technical advantages as a macromolecular substance produced by microorganisms, so it has a good application prospect in food, medicine and related fields.

### BRIEF DESCRIPTION OF THE DRAWINGS

FIG. 1 is a gel filtration chromatography elution curve of a crude CGMCC No. 8333 extracellular polysaccharide.

FIG. 2 is a high performance gel filtration chromatogram of a CGMCC No. 8333 extracellular polysaccharide.

FIG. 3 is a 1H-NMR chromatogram of the CGMCC No. 8333 extracellular polysaccharide.

FIG. 4 is a 13C-NMR chromatogram of the CGMCC No. 8333 extracellular polysaccharide.

FIG. 5 is an H-H COSY chromatogram of the CGMCC No. 8333 extracellular polysaccharide.

FIG. 6 is an HSQC chromatogram of the CGMCC No. 8333 extracellular polysaccharide.

FIG. 7 is a TOCSY chromatogram of the CGMCC No. 8333 extracellular polysaccharide.

FIG. 8 is HMBC chromatogram 1 of the CGMCC No. 8333 extracellular polysaccharide.

FIG. 9 is HMBC chromatogram 2 of the CGMCC No. 8333 extracellular polysaccharide.

FIG. 10 is an NOESY chromatogram of the CGMCC No. 8333 extracellular polysaccharide.

FIG. 11 shows an effect of the CGMCC No. 8333 extracellular polysaccharide on RAW264.7 cells.

FIG. 12 shows an effect of the CGMCC No. 8333 extracellular polysaccharide on a phagocytic ability of the RAW264.7 cells.

FIG. 13 shows an effect of the CGMCC No. 8333 extracellular polysaccharide on release of NO from the RAW264.7 cells.

FIG. 14 shows an effect of the CGMCC No. 8333 extracellular polysaccharide on secretion of TNF-α by the RAW264.7 cells.

FIG. 15 shows an effect of the CGMCC No. 8333 extracellular polysaccharide on secretion of IL-1(3 by the RAW264.7 cells.

FIG. 16 shows an effect of the CGMCC No. 8333 extracellular polysaccharide on secretion of IL-6 by the RAW264.7 cells.

### DETAILED DESCRIPTION OF THE EMBODIMENTS

A strain of Paenibacillus sp. for fermenting wheat bran to synthesize an extracellular polysaccharide, wherein a preservation number of the strain is CGMCC NO.8333.

The Paenibacillus sp. of the present invention is isolated from kefir, and has been identified with phenotypical as well as phylogentical characteristics. The cell shapes and physical and chemical experimental results of the disclosed bacterial strain are shown in the following table.

| Test project | Result | Test project | Result | Test project | Result |
|---|---|---|---|---|---|
| Cell shape | Rod shape | Gram staining | Positive | Spore formation | + |
| Oxidase | − | Catalase | + | β-galactosidase | + |
| Arginine | − | Lysine | − | Ornithine | − |
| dihydrolase | | decarboxylase | | decarboxylase | |
| Urease | − | Citrate | − | Nitrate reduction | + |
| | | utilization | | | |
| Indole generation | − | VP reaction | + | H₂S generation | − |
| Starch | + | Esculin | + | Gelatin | + |
| hydrolysis | | hydrolysis | | liquefaction | |

Acid production from carbohydrates

| | | | | | |
|---|---|---|---|---|---|
| Control | − | D-galactose | + | Melibiose | + |
| Glycerol | − | D-glucose | + | Gluconate | + |
| Erythritol | − | D-fructose | + | N-acetyl-glucosamine | + |
| D-arabinose | − | D-mannose | + | Amygdalin | + |
| L-arabinose | + | L-sorbose | − | Arbutin | + |
| D-ribose | + | L-rhamnose | − | Esculin | + |
| D-xylose | + | Dulcitol | − | Salicin | + |
| L-xylose | − | Inositol | − | Cellobiose | + |
| Adonitol | − | Mannitol | + | Maltose | + |
| β-methyl-D-xyloside | + | Sorbitol | − | Lactose | + |
| α-methyl-D-mannoside | − | D-lyxose | − | D-fucose | − |
| α-methyl-D-glucoside | − | D-arabitol | − | L-arabitol | − |
| 2-keto-gluconate | + | L-fucose | − | Gentiobiose | + |
| 5-keto-gluconate | − | Xylitol | − | Starch | + |
| Sucrose | + | Melezitose | − | Glycogen | + |
| D-tagatose | − | Raffinose | + | | |

16SrRNA gene sequence determination results are as follows:

(SEQ ID NO: 1)
TGCAGTCGAGCGGAGTTGATAGAGTGCTTGCACTCTTGAGACTTAGCGG
CGGACGGGTGAGTAACACGTAGGCAACCTGCCCCTCAGACTGGGATAAC
TACCGGAAACGGTAGCTAATACCGGATAATCGTTTTCTTCTCCTGAAGA
GACCGGGAAAGACGGAGCAATCTGTCACTGAGGGATGGGCCTGCGGCGC
ATTAGCTAGTTGGTGGGGTAACGGCTCACCAAGGCGACGATGCGTAGCC
GACCTGAGAGGGTGATCGGCCACACTGGGACTGAGACACGGCCCAGACT
CCTACGGGAGGCAGCAGTAGGGAATCTTCCGCAATGGACGAAAGTCTGA
CGGAGCAACGCCGCGTGAGTGATGAAGGTTTTCGGATCGTAAAGCTCTG
TTGCCAGGGAAGAACGTCTTCTAGAGTAACTGCTAGAAGAGTGACGGTA
TCCGGAATTATTGGGCGTAAAGCGCGCGCAGGCGGTCATTTAAGTCTGG
TCCTGAGAAGAAAGCCCCGGCTAACTACGTGCCAGCAGCCGCGGTAATA
CGTAGGGGGCAAGCGTTGGTTTAATCCCGAAGCTCAACTTCGGGTCGCA
TCGGAAACTGGATGACTTGAGTGCAGAAGAGGAGAGTGGAATTCCACGT
GTAGCGGTGAAATGCGTAGAGATGTGGAGGAACACCAGTGGCGAAGGCG
ACTCTCTGGGCTGTAACTGACGCTGAGGCGCGAAAGCGTGGGGAGCAAA
CAGGATTAGATACCCTGGTAGTCCACGCCGTAAACGATGAATGCTAGGT
GTTAGGGGTTTCGATACCCTTGGTGCCGAAGTTAACACATTAAGCATTC
CGCCTGGGGAGTACGGTCGCAAGACTGAAACTCAAAGGAATTGACGGGG
ACCCGCACAAGCAGTGGAGTATGTGGTTTAATTCGAAGCAACGCGAAGA
ACCTTACCAGGTCTTGACATCTGAATGACCGGTGCAGAGATGTACCTTT
TCTTCGGAACATTCAAGACAGGTGGTGCATGGTTGTCGTCAGCTCGTGT
CGTGAGATGTTGGGTTAAGTCCCGCAACGAGCGCAACCCTTATGCTTAG
TTGCCAGCACATCATGGTGGGCACTCTAAGCAGACTGCCGGTGACAAAC
CGGAGGAAGGTGGGGATGACGTCAAATCATCATGCCCCTTATGACCTGG
GCTACACACGTACTACAATGGTCGGTACAACGGGAAGCGAAGCCGCGAG
GTGGAGCGAATCCTAAAAAGCCGATCTCAGTTCGGATTGCAGGCTGCAA
CTCGCCTGCATGAAGTCGGAATTGCTAGTAATCGCGGATCAGCATGCCG
CGGTGAATACGTTCCCGGGTCTTGTACACACCGCCCGTCACACCACGAG
AGTTTGCAACACCCGAAGTCGGTGGGGTAACCCGCAAGGAGCCAGCCGC
CGAAGGTGGTCAGT

The strain was preserved in the China General Microbiological Culture Collection Center (CGMCC) on Oct. 14, 2013. The preservation address is: No. 3 Courtyard 1 West Beichen Road, Chaoyang District, Beijing 100101, China.

The Paenibacillus sp. disclosed in the present invention may ferment the wheat bran to synthesize the extracellular polysaccharide. At present, microbial polysaccharides are generated by microorganisms metabolizing carbohydrates. Usually, the cultivation medium for microbial polysaccharide synthesis is usually a chemically defined or semi-defined one, such as MRS, TYC, etc. However, relatively fewer studies have been done on the synthesis of the microbial polysaccharides by using natural materials, especially agriculture by-products (such as the wheat bran). In addition, as far as fermenting strains are concerned, research on strains that can ferment bran to synthesize an extracellular polysaccharide is also relatively scarce. In the present invention, the Paenibacillus sp. can not only ferment the wheat bran, but also synthesize an extracellular polysaccharide with specific structure and functionality.

The weight-average molecular weight of the extracellular polysaccharide synthesized by the Paenibacillus sp. CGMCC NO.8333 of the present invention is 300,800 to 451,200 daltons, and the extracellular polysaccharide is an acidic heteropolysaccharide composed of glucuronic acid, glucose and fucose in a molar ratio of (1.55 to 1.60): 1:(1.63 to 1.72). The backbone of the extracellular polysaccharide is composed of a 1,3-linked glucose residue, a 1,3-linked fucose residue, a 1,3,4-linked fucose residue and a 1,4-linked glucuronic acid residue, a branch point is located at a 0-4 position of the 1,3,4-linked fucose residue, a branch chain is composed of terminally linked glucuronic acid residues, and the extracellular polysaccharide is composed of a repeating unit shown in Formula I.

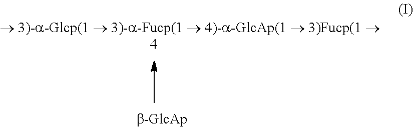

The present invention further provides use of an extracellular polysaccharide synthesized by a strain of Paenibacillus sp. in fields of food and medicine. Research has shown that microbial extracellular polysaccharides have biological activities, such as immune activity, tumor resisting, oxidation resisting and regulation of intestinal flora, etc., and may be used in the fields of food and medicine. Immunomodulation is demonstrated by polysaccharides with specific chemical structures, some of them might be utilized as a non-specific immunomodulator. An immune system is a body's defense system to resist invasion of pathogenic microorganisms and remove mutant cells in a body, and is mainly composed of immune organs, immune cells and immune molecules. In a normal body, the immune system maintains a dynamic balance to keep the body stable. However, when the immune system is disturbed, a series of diseases will be induced, which will impair normal physiological functions of the body. Research has found that the immunomodulatory effects of the extracellular polysaccharides on the body mainly include activating macrophages, activating immune cells such as B cells and T cells, entering cells through cell surface receptor recognition to improve the phagocytic and secretion abilities of cells, activating complements, activating a reticuloendothelial system and promoting the development of the immune organs. So far, a few of extracellular polysaccharides have been disclosed for their activity in immunomodulation. In the present invention, the extracellular polysaccharide synthesized by the Paenibacillus sp. CGMCC NO.8333 has no cytotoxicity when a concentration is lower than 100 μg/mL, can activate RAW264.7 cells, enhance the phagocytic ability thereof, and improve expression of NO, TNF-α, IL-1 and IL-6 cytokines by activating the RAW264.7 cells, and has a good immunomodulatory effect.

The present invention further provides use of an extracellular polysaccharide synthesized by a strain of Paenibacillus sp. in preparation of immunomodulatory agents.

The above specific implementations are further described below through the embodiments, but this does not limit the present invention to the scope of the described embodiments. Experimental methods that do not specify specific conditions in the following embodiments are selected according to conventional methods and conditions, or according to product specifications.

In the following embodiments, all raw materials are commercially available and meet relevant national standards.

### Embodiment 1 Preparation of Paenibacillus sp. Extracellular Polysaccharide

1. Materials and Methods

(a) Preparation of seeds (fermenting strains): lyophilized Paenibacillus sp. CGMCC No. 8333 was resuspended in a small amount of sterile distilled water, and a loop thereof was streaked onto a plate containing 1% (w/w) skim milk and 1.5% (w/v) agar (purchased from Sinopharm Group, China), cultivated aerobically for 48 hours at 30° C. A single colony was picked by an inoculating loop to be inoculated into a flask containing 10 mL of sterile 10% (w/w) skim milk, and cultivated for 24 hours at 30° C. at 180 rpm to obtain the seeds for fermenting.

(b) Preparation of a wheat bran culture medium: 1.8 g of wheat bran (commercially available) and 58.2 mL of distilled water were added to a 250 mL conical flask to be mixed evenly, heated and boiled, then sterilized for 15 minutes at 110° C., and cooled to room temperature to obtain the required wheat bran culture medium.

2. Preparation of Crude Paenibacillus sp. Extracellular Polysaccharide

The Paenibacillus sp. CGMCC No. 8333 seeds were aseptically inoculated into the above wheat bran culture medium at a ratio of 3% (v/v, a volume percentage of a seed liquid to a fermentation broth, the same below), and subjected to shake culture for 48 hours at 26° C. at 180 rpm to obtain the fermented broth. The fermented broth was centrifuged for 10 minutes at 15,000 rpm. The supernatant was taken, heated and boiled for 10 minutes, and cooled to room temperature. Three volumes of absolute ethyl alcohol (purchased from Sinopharm Group, China) was slowly added to the heat treated supernatant. The mixture was stored under refrigeration for 24 hours, and centrifuged for 10 minutes at 15,000 rpm. The precipitate was redissoved completely with a small amount of distilled water, and then lyophilized to obtain crude Paenibacillus sp. extracellular polysaccharide A.

3. Preparation of Paenibacillus sp. Extracellular Polysaccharide (Single Component)

250 mg of the crude Paenibacillus sp. extracellular polysaccharide A was dissolved into 25 mL of a Tris-HCl buffer solution (50 mM, pH 7.6) (purchased from Sinopharm Group, China), loaded onto a pre-equilibrated DEAE-Sepharose Fast Flow (purchased from GE Company, USA) column by using a constant flow pump, subjected to isocratic elution with a Tris-HCl buffer solution (50 mM, pH 7.6) and the same Tris-HCl buffer solution containing 0.2 mol/L NaCl or 0.4 mol/L NaCl in sequence, wherein an elution speed was 1.5 mL/min. The eluant (8 mL per tube) was collected with an automatic sampling instrument (purchased from Shanghai Qingpu-Huxi Instruments Factory, China).

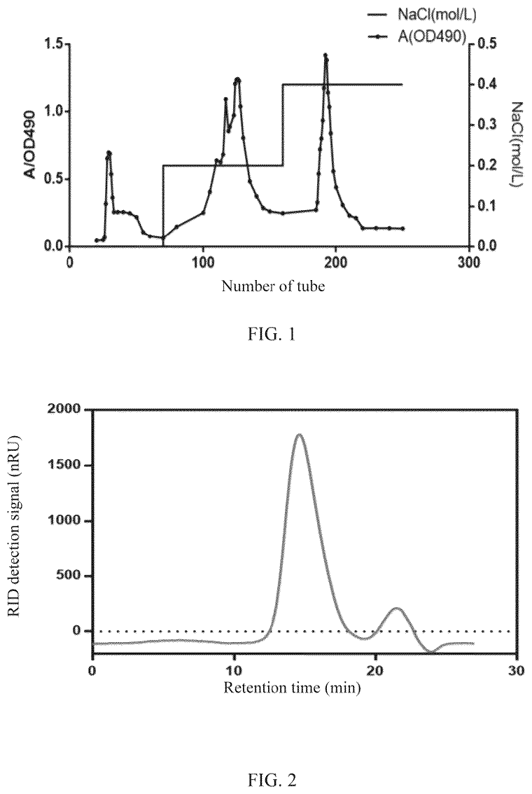

The content of polysaccharides in each tube of eluate was detected by using a sulfuric acid-phenol method. The eluate in tubes containing the third component peak was combined and collected (tubes 185 to 205 in FIG. 1, an abscissa represents the numbers of tubes, and an ordinate represents absorbance values), loaded into a dialysis bag with a molecular weight cut-off of 14,000 daltons, dialyzed against deionized water for 72 hours to remove the buffer salt, changing the water every 12 hours, and lyophilized to obtain a single-component polysaccharide, namely the Paenibacillus sp. extracellular polysaccharide of the present invention, called EPS-1.

### Embodiment 2 Preparation of Paenibacillus sp. Extracellular Polysaccharide

1. Materials and Methods

(a) Preparation of seeds (fermenting strains): the same as in Embodiment 1.

(b) Preparation of a wheat bran culture medium: 0.6 g of wheat bran and 59.4 mL of distilled water were added to a 250 mL conical flask to be mixed evenly, heated and boiled, then sterilized for 5 minutes at 125° C., and cooled to a room temperature to obtain the required wheat bran culture medium.

2. Preparation of Crude Paenibacillus sp. Extracellular Polysaccharide

The Paenibacillus sp. CGMCC No. 8333 seeds were aseptically inoculated into the above wheat bran culture medium with an inoculation ratio of 5%, and cultivated for 12 hours at 37° C. at 300 rpm to obtain the fermented broth. The fermented broth was centrifuged for 10 minutes at 15,000 rpm. The supernatant was taken, heated and boiled for 10 minutes, and cooled to room temperature. Three volumes of absolute ethyl alcohol was slowly added to the heat treated supernatant. The mixture was stored under refrigeration for 24 hours, and centrifuged for 10 minutes at 15,000 rpm. The precipitate was redissolved completely with a small amount of distilled water, and then lyophilized to obtain crude Paenibacillus sp. extracellular polysaccharide B.

3. Preparation of Paenibacillus sp. Extracellular Polysaccharide (Single Component)

With reference to the method in Embodiment 1, the Paenibacillus sp. extracellular polysaccharide B was separated to obtain single-component extracellular polysaccharide EPS-2 of Paenibacillus sp..

### Embodiment 3 Preparation of Paenibacillus sp. Extracellular Polysaccharide

1. Materials and methods

(a) Preparation of seeds (fermenting strains): the same as in Embodiment 1.

(b) Preparation of a wheat bran culture medium: 3 g of wheat bran and 57 mL of distilled water were added to a 250 mL conical flask to be mixed evenly, heated and boiled, then sterilized for 25 minutes at 95° C., and cooled to room temperature to obtain the required wheat bran culture medium.

2. Preparation of Crude Paenibacillus sp. Extracellular Polysaccharide

The Paenibacillus sp. CGMCC No. 8333 seeds were aseptically inoculated into the above wheat bran culture medium at a ratio of 1%, and cultivated for 72 hours at 20° C. at 100 rpm to obtain the fermented broth. The fermented broth was centrifuged for 10 minutes at 15,000 rpm. The supernatant was taken, and boiled for 10 minutes, and cooled to room temperature. Three volumes of absolute ethyl alcohol was slowly added to the heat treated supernantant. The mixture was stored under refrigeration for 24 hours, and centrifuged for 10 minutes at 15,000 rpm. The precipitate was redissolved completely with a small amount of distilled water, and then lyophilized to obtain crude Paenibacillus sp. extracellular polysaccharide C.

3. Preparation of Paenibacillus sp. Extracellular Polysaccharide (Single Component)

With reference to the method in Embodiment 1, the Paenibacillus sp. extracellular polysaccharide C was separated to obtain single-component extracellular polysaccharide EPS-3 of Paenibacillus sp..

### Embodiment 4 Verification of the Purity of Paenibacillus sp. Extracellular Polysaccharide

5 mg of EPS-1, EPS-2 and EPS-3 samples were dissolved into 5 mL of ultrapure water, respectively, to prepare a 1 mg/mL polysaccharide solution, which was subjected to a 0.45 μm filter membrane, and then injected into an Agilent 1100 high performance liquid chromatograph (Agilent Company, USA) for analysis, wherein chromatographic conditions were as follows: an RID detector, a chromatographic column was TSK-Gel G6000PWXL (purchased from Tosoh (Shanghai) Bioscience Co., Ltd., Japan), and a mobile phase is 0.1 mol/L NaNO₃ solution. A flow rate was set to be 0.6 mL/min, a column temperature was 35° C., and an injection volume was 20 μL. The purity (unity) thereof was judged according to peak shapes.

In particular, the high performance gel filtration chromatogram of the EPS-1 sample was shown in FIG. 2 (the chromatograms of the EPS-2 and EPS-3 are similar). A symmetrical chromatographic peak was shown at retention time of 14 to 15 minutes, and a chromatographic peak at 21 minute was a mobile phase peak. The above results shown that the EPS-1, EPS-2 and EPS-3 samples were all homogeneous polysaccharide components.

### Embodiment 5 Determination of Molecular Weights of Paenibacillus sp. Extracellular Polysaccharide

Glucan with different molecular weights was used as standards: STD-1 (Mw=5,000), STD-2 (Mw=12,000), STD-3 (Mw=50,000), STD-4 (Mw=270,000), STD-5 (Mw=670,000). The above series of standard polysaccharides and the EPS-1, EPS-2 and EPS-3 samples were dissolved into a mobile phase (a 0.1 mol/L NaNO₃ solution) respectively to obtain a 1 mg/mL solution, which was individually filtered with a 0.45 μm filter membrane, and then analyzed by an Agilent 1100 high performance liquid chromatograph. The chromatographic conditions were the same as those in Embodiment 4. A standard curve was drawn by taking logarithms LgMw of the molecular weights of the standard polysaccharides as abscissas and taking the retention time tR as ordinates, to obtain a linear regression equation between the logarithms of the molecular weights and the retention time. The molecular weights of the extracellular polysaccharide samples could be calculated according to the regression equation, and results were shown in the table below.

TABLE 1
Determination of molecular weights of Paenibacillus
sp. extracellular polysaccharide

| | Extracellular polysaccharide samples | | |
|---|---|---|---|
| | EPS-1 | EPS-2 | EPS-3 |
| Weight-average | 376,000 | 300,800 | 451,200 |
| molecular weight | | | |
| (Daltons) | | | |

Conclusion: the weight-average molecular weight of the Paenibacillus sp. extracellular polysaccharide was 300,800 to 451,200 daltons.

### Embodiment 6 Determination of Monosaccharide Compositions of Paenibacillus sp. Extracellular Polysaccharide

(1) Hydrolysis of Polysaccharide Samples

2.0 mg of EPS-1, EPS-2 and EPS-3 samples were taken to be placed into ampoules respectively. 3 mL of 2 mol/L trifluoroacetic acid (TFA) was added. After sealing, they were hydrolyzed for 5 hours at 110° C. After cooling, hydrolysates were subjected to rotary evaporation under reduced pressure to dryness at 45° C., methanol was added to continue rotary evaporation, and the above operations were repeated several times to remove excess TFA to obtain EPS hydrolysates.

(2) Derivatization of Hydrolyzed Samples and Mixed Monosaccharide Standard Samples

The above EPS hydrolysis products were dissolved into 1 mL of water to obtain sample solutions to be derivatized. 1 mL of the above sample solutions or a mixed standard solution of nine monosaccharides (0.5 mg/mL, rhamnose, fucose, glucuronic acid, galactose, glucose, mannose, galacturonic acid, arabinose and xylose) were taken. 1 mL of a 0.6 mol/L NaOH solution and 1 mL of a 0.5 mol/L PMP methanol solution were added. Even mixing was conducted to completely dissolve a solid product. Derivatization was conducted for 100 minutes in a 70° C. oven. After cooling to room temperature, 0.3 mol/L HCl was added dropwise to adjust to be neutral. After extracting three times with trichloromethane, an aqueous phase was collected. The aqueous phase was filtered with a 0.45 μm filter membrane, and then used for HPLC analysis.

(3) Chromatographic Conditions

An Agilent 1260 high performance liquid chromatograph (Agilent Company, USA) was adopted. A DAD detector was equipped. A chromatographic column was an Agilent Eclipse XDB-C18 column (purchased from Agilent Company, USA). The column temperature was kept at 30° C. An injection volume was 20 μL. The mobile phase was acetonitrile to 0.1 mol/L phosphate buffer solution (pH 6.8) at 16:84(V/V). A detection wavelength was 250 nm.

(4) Data Analysis

The the monosaccharide compositions of the polysaccharide samples were determined with reference to the retention time of different monosaccharide standard samples (purchased from sigma Company, USA). Then, the molar ratio of the individual monosaccharide in the polysaccharide samples was determined according to a peak area ratio of the respective monosaccharide compositions. Results were shown in Table 2.

TABLE 2
A molar ratio of monosaccharides of a Paenibacillus
sp. extracellular polysaccharide

| | Glucuronic acid | Glucose | Fucose |
|---|---|---|---|
| EPS-1 (in a molar ratio) | 1.58 | 1 | 1.66 |
| EPS-2 (in a molar ratio) | 1.55 | 1 | 1.63 |
| EPS-3 (in a molar ratio) | 1.60 | 1 | 1.72 |

Conclusion: the Paenibacillus sp. extracellular polysaccharide was an acidic heteropolysaccharide composed of glucuronic acid, glucose and fucose in a molar ratio of (1.55 to 1.60):1:(1.63 to 1.72).

### Embodiment 7 Determination of Linkage Mode of Paenibacillus sp. Extracellular Polysaccharide

(1) Methylation Analysis

Uronic acid reduction: a uronic acid was reduced by the carbodiimide-sodium borohydride method (EDC-NaBH₄). The reaction was divided into two stages. In the first stage: 50 mg of the EPS-1 was dissolved into 6 mL of ultrapure water. 500 mg of EDC was added into a solution twice, with an interval time of 30 minutes, and 0.1 mol/L HCl was used to control the pH value of the solution to be 4.5 to 4.8. The whole reaction process lasted for 3 hours. In the second stage: 8 mL of 2 mol/L NaBH₄ was added dropwise into the above system within 40 minutes, and the pH of the mixture was controlled at 7.0. After the addition of NaBH₄ was completed, the reaction was continued for 1 hour, and the obtained mixture was placed in a dialysis bag (a molecular weight cut-off of 3500 Da) for dialysis against running water for 24 hours. The above steps were repeated four times, and whether the uronic acid was reduced completely was detected by PMP-HPLC.

Methylation: 20 mg of the polysaccharide sample whose uronic acid has been completely reduced was placed in a 10 mL reaction flask, and 3 mL of anhydrous dimethyl sulfoxide was quickly added at room temperature. Sealing was conducted. Magnetic stirring was conducted for 30 minutes, and assistance of ultrasonic wave was used to dissolve the sample. Then, 50 mg of dry NaOH powder was quickly added. The mixture was stirred under sealing until most of the NaOH was dissolved, and then ice bathed for 5 minutes. 1 mL of methyl iodide was slowly added dropwise within 30 minutes. Stirring was conducted at room temperature in dark further for 30 minutes, and finally 1 mL of ultrapure water was added to terminate the reaction. The methylated product was placed in a dialysis bag for dialysis against running water for 24 hours, and subjected to rotary evaporation to dryness. Then, the above steps were repeated. After multiple times of methylation, a small amount of samples was taken for infrared spectrum detection. If an O—H stretching vibration absorption peak of the polysaccharide sample at 3400 to 3000 cm-1 disappeared, it was indicated that the polysaccharide sample had been completely methylated; if the sample had not been completely methylated, it was necessary to continue the reaction until the polysaccharide sample was completely methylated.

Hydrolysis and acetylation: 2 mg of the completely methylated EPS-1 sample was placed in an ampoule, and then 3 mL of 2 mol/L TFA was added and the ampoule was sealed. The hydrolysis was conducted for 4 hours at 110° C. Methanol was added for rotary evaporation under reduced pressure several times to completely remove the TFA. After rotary dryness, the obtained solid was dissolved with 3 mL of ultrapure water and 50 mg of NaBH₄ was added. The mixture was stirred magnetically at room temperature for 3 hours. After the reaction was completed, acetic acid was added until the solution was weakly acidic (pH=5), methanol was added, and rotary evaporation to dryness was conducted. The above steps were repeated several times to fully remove boric acid. The obtained solid was dried for 10 minutes in a 100° C. oven. 3 mL of acetic anhydride was added. Reaction was conducted for 100 minutes at 100° C. After the reaction was completed, toluene (3 mL) was added for co-evaporation several times to remove excess acetic anhydride. The solid was dissolved into trichloromethane (5 mL), and extracted 3 times with ultrapure water (5 mL×3). The trichloromethane layer was recovered and anhydrous sodium sulfate powder was added to remove water. The mixture was set for 30 minutes. The trichloromethane phase was evaporated under reduced pressure to dryness. The obtained solid was disssolved with 0.5 mL of the trichloromethane and filtrated with a 0.22 μm organic filter membrane, and then GC-MS analysis was conducted.

GC-MS conditions: an instrument model was Agilent 7820 A/5977 GC-MS (purchased from Agilent Company, USA); a chromatographic column model was an HP-5 capillary column; temperature programming was as follows: an initial temperature was 120° C. and maintained for 2 minutes and then increased to 250° C., a temperature increasing rate was 5° C./min, and the temperature was maintained for 10 minutes; an injection port was in a splitting mode, and a splitting ratio was 3:1; an injection volume was 1 μL. A mass spectrometry ion source was an EI source, a voltage of the ion source was 70 eV, and a temperature was 180° C.

Data analysis: an ELMS profile obtained by GC-MS was compared with a standard PMAA map, and in combination with results of monosaccharide compositions, it could be determined that a linkage mode of respective monosaccharide residues in the EPS-1 after reduction is a 1,3-linked fucose residue, a 1,3,4-linked fucose residue, a 1,3-linked glucose residue, a 1,4-linked glucuronic acid residue, and terminally linked glucuronic acid residues.

(2) Nuclear Magnetic Resonance Spectrum Analysis

20 mg of the EPS-1 sample was taken, dissolved with 0.5 mL of D20, transferred to a clean nuclear magnetic resonance tube, and subjected to chromatographic analysis on a 600 MHz nuclear magnetic resonance instrument (purchased from Bruker Company, Switzerland).

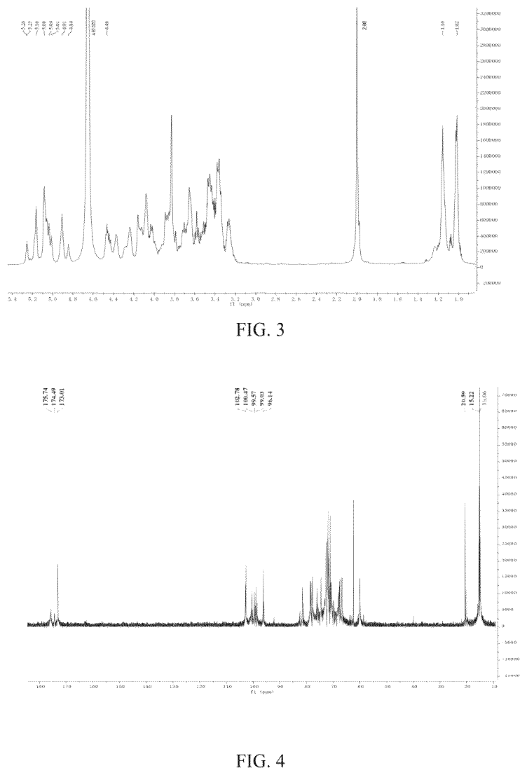

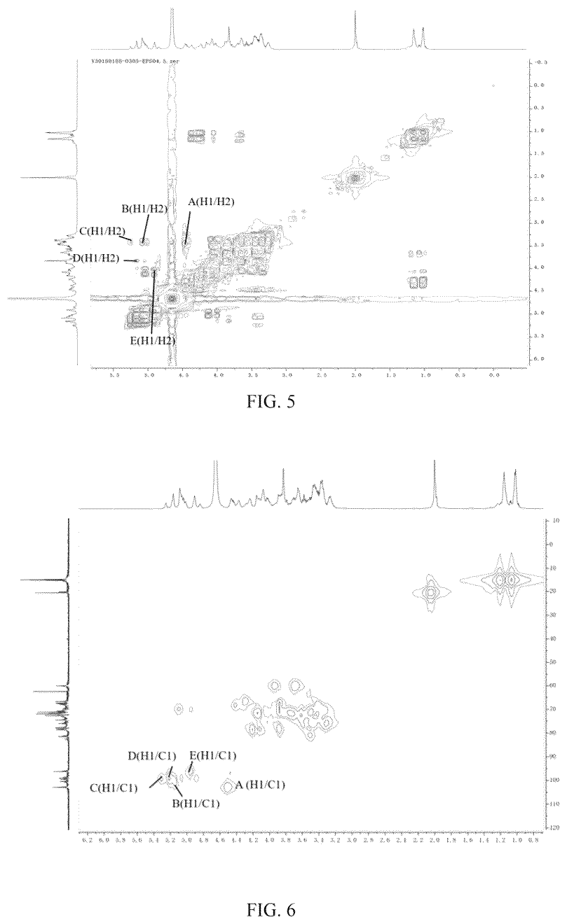

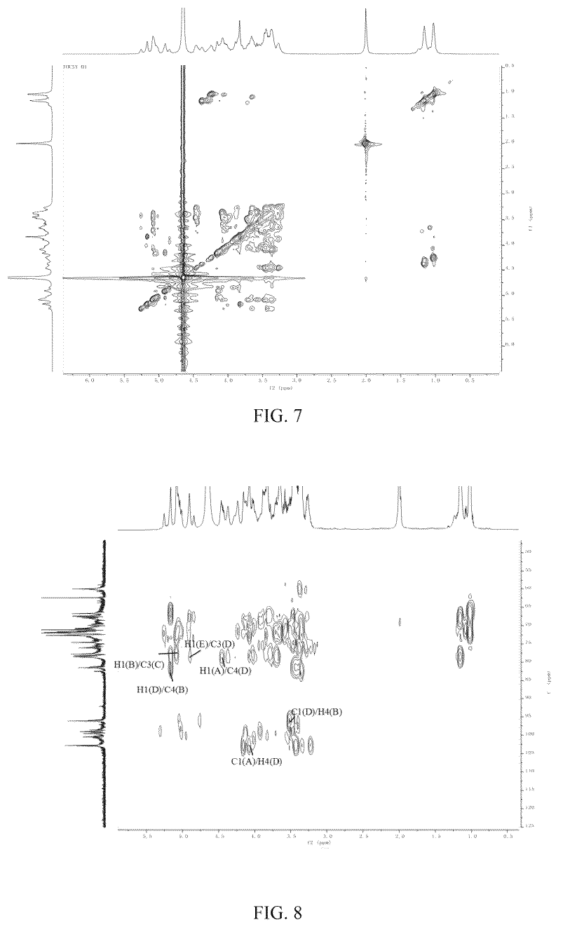

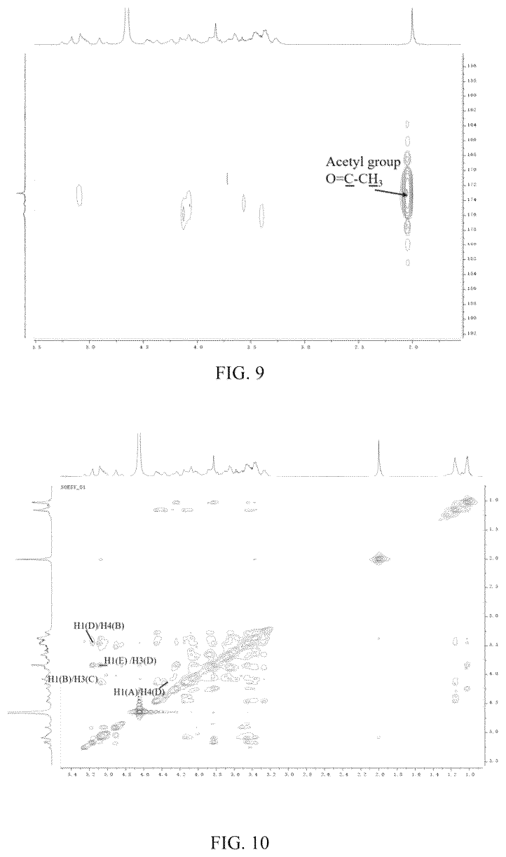

After comprehensive chromatographic analysis on ¹H-NMR (FIG. 3), ¹³C-NMR (FIG. 4), H-H COSY (FIG. 5), HSQC (FIG. 6), TOCSY (FIG. 7), HMBC (FIG. 8, FIG. 9) and NOESY (FIG. 10), it was found that a backbone of the EPS-1 was composed of the 1,3-linked glucose residue, the 1,3-linked fucose residue, the 1,3,4-linked fucose residue and the 1,4-linked glucuronic acid residue, a branch point was located at a 0-4 position of the 1,3,4-linked fucose residue, and a branch chain was composed of the terminally linked glucuronic acid residues.

Based on the foregoing, the Paenibacillus sp. extracellular polysaccharide was composed of a repeating unit shown in Formula I.

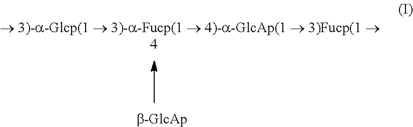

### Effect Embodiment 1 Effect of Paenibacillus sp. Extracellular Polysaccharide on RAW264.7 Cell Growth

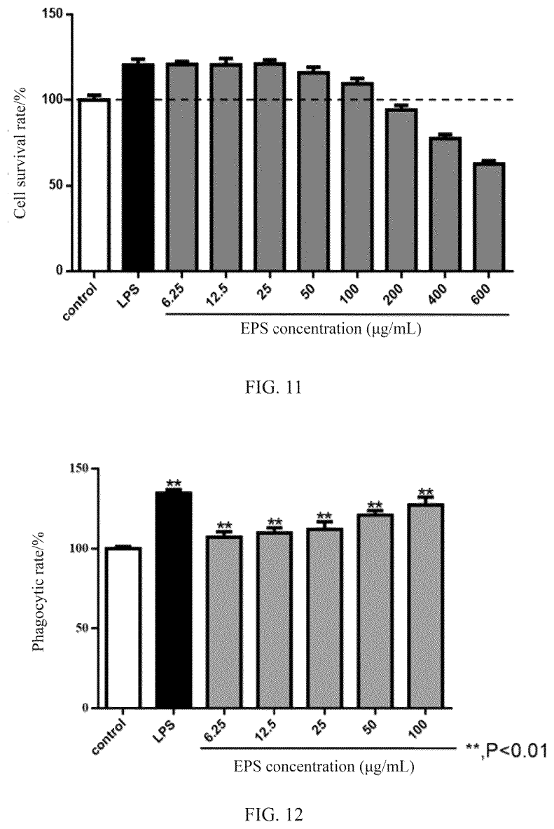

The RAW264.7 cells was adjusted to 1×10⁴ cells/mL and inoculated in a 96-well microplate with 200 μL/well, and cultivated in humified atomsphere supplemented with 5% CO₂ at 37° C. After cell adherence, the culture supernatant was discarded. 200 μL EPS-1 solutions of different concentrations (0, 6.25, 12.5, 25, 50, 100, 200, 400, 600 μg/mL) were added respectively, employing LPS (1 μg/mL) as a positive control, with five parallel wells for each concentration. After culturing for 24 hours, the liquid in the well was sucked out. 30 μL of a sterilized MTT solution (5 mg/mL) was added to each well. After incubation for 4 hours, the liquid in the well was sucked out. 200 μL of DMSO was added to each well. After purple crystals in the well plate were completely dissolved by shaking, the optical density (OD) was measured with a microplate reader (purchased from Molecular Devices, USA) at 492 nm, and the survival rate of the cells treated with EPS-1 was calculated by the following formula, and results were shown in FIG. 11.

Survival rate %=OD_{experimental group}/OD_{control group}×100%

When the concentrations of the EPS-1 were 6.25, 12.5, 25, 50 and 100 μg/mL respectively, survival rates of the RAW264.7 cells were all higher than those in a blank control group, which were 120.92±1.73%, 120.637±3.80%, 121.15±2.37%, 116.05±3.24% and 109.70±3.16%, respectively; when an administration concentration was increased to 200 μg/mL, the survival rates of the RAW264.7 cells were reduced and lower than those in the blank control group; when the administration concentration reached the maximum concentration, namely 600 μg/mL, the survival rates of the RAW264.7 cells were significantly lower than those in the blank control group, and were only 62.74±2.94%. The above experimental results showed that the EPS-1 had no cytotoxicity to the RAW264.7 cells within the range of 6.25 to 100 μg/mL.

Conclusion: when the concentration was lower than 100 μg/mL, the Paenibacillus sp. extracellular polysaccharide showed no cytotoxicity to the RAW264.7 cells.

### Effect Embodiment 2 Effect of Paenibacillus sp. Extracellular Polysaccharide on Phagocytic Activity of RAW264.7 Cells

The RAW264.7 cells was adjusted to 1×10⁴ cells/mL and inoculated in a 96-well microplate with 200 μL/well, and cultivated in humified atomsphere supplemented with 5% CO₂ at 37° C. After cell adherence, the fluid was discarded. EPS-1 solutions of different concentrations (0, 6.25, 12.5, 25, 50 and 100 μg/mL) and 100 μL of LPS (1 μg/mL) were added to each well respectively, and five parallel wells were set for each concentration. After culturing for 24 hours, a 0.08% freshly prepared neutral red solution was added. Incubation was conducted for 1 hour in an incubator and then the fluid was sucked out. The cells in the well bottom were rinsed twice with PBS and the fluid was dropped off. A lysate solution (glacial acetic acid:ethanol=1:1) was added for lysis for 1 hour. An optical density was measured with a microplate reader at 492 nm. Phagocytic rates of RAW264.7 cells treated with varied levels of EPS-1 were calculated by the following formula, and results were shown in FIG. 12.

Phagocytic rate %=OD_{experimental group}/OD_{control group}×100%

At the concentrations of 6.25, 12.5, 25, 50 and 100 μg/mL, the phagocytic rates of RAW264.7 cells treated with EPS-1 were 105.63±2.17%, 109.11±2.43%, 109.91±2.75%, 119.42±1.84%, and 126.25±2.77% respectively, and all significantly higher than those in a blank control group. Within the above concentration range, as the concentration of EPS-1 increased, the phagocytic ability of the RAW264.7 cells was gradually enhanced, and the phagocytic rate of the cells treated with EPS-1 at 100 μg/mL was close to that in a positive control group (129.03±3.13%).

Conclusion: the Paenibacillus sp. extracellular polysaccharide could activate the RAW264.7 cells between 6.25 Ng/mL and 100 Ng/mL and enhance their phagocytic ability.

### Effect Embodiment 3 Effect of Paenibacillus sp. Extracellular Polysaccharide on Release of Cytokines from RAW264.7 Cells

The RAW264.7 cells was adjusted to 5×10⁵ cells/mL and inoculated in a 96-well microplate with 200 μL/well, and cultivated in humidified atmosphere supplemented with 5% CO₂ at 37° C. After cell adherence, the fluid was discarded. 100 μL of EPS-1 solutions of different concentrations (0, 6.25, 12.5, 25, 50, 100, 200, 400 and 600 μg/mL) and 100 μL of LPS (1 μg/mL) were added to each well respectively, culturing was conducted for 24 hours, and two parallel wells were set for each concentration. 50 μL of the supernatant was collected from each well, and cytokines released were detected by an NO kit, a TNF-α ELISA kit, an IL-1β ELISA kit and an IL-6 ELISA kit (purchased from ELISA Company, USA).

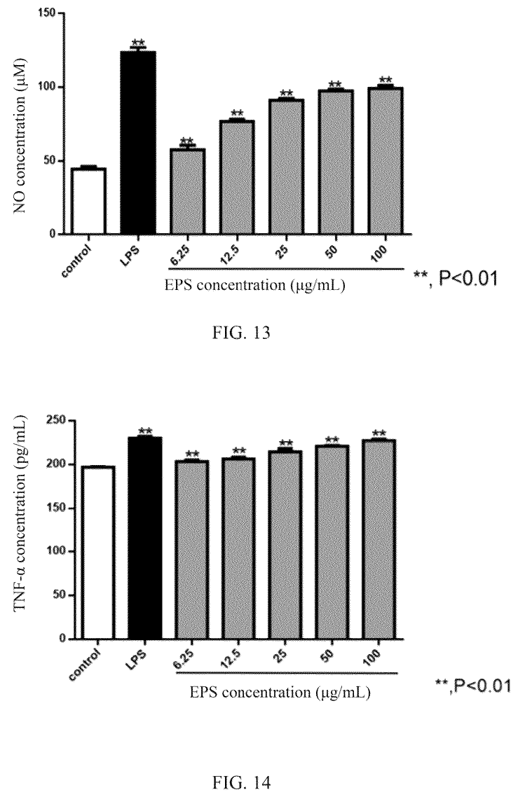

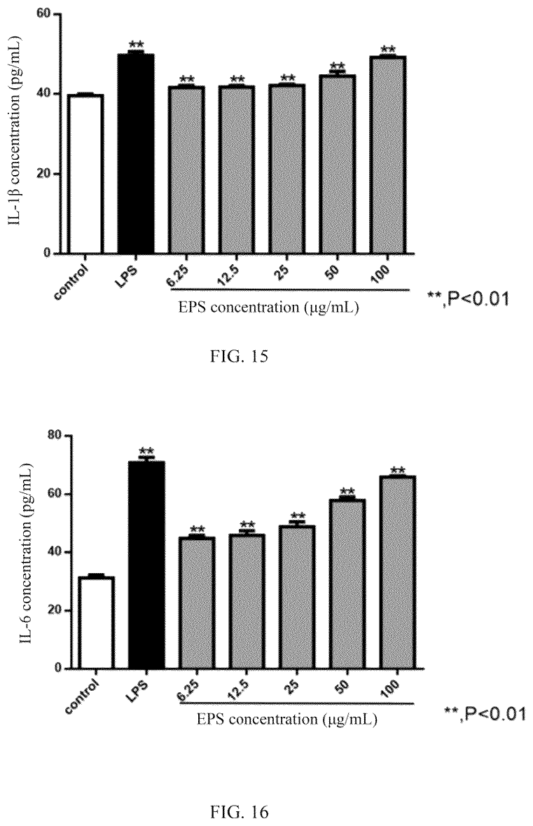

Results were shown in FIG. 13 (NO), FIG. 14 (TNF-α), FIG. 15 (IL-1β) and FIG. 16 (IL-6). The release of NO, TNF-α, IL-1β and IL-6 in RAW264.7 was increased in a dose-dependent manner in the presence of EPS-1, indicating that the Paenibacillus sp. extracellular polysaccharide could stimulate the RAW264.7 cells to secrete a variety of cytokines and exert its immunomodulatory effects.

The bacterial strain that can ferment the wheat bran to synthesize the extracellular polysaccharide provided by the present invention is described above in detail. The principles and implementations of the present invention are described herein by applying specific examples, and the descriptions of the above embodiments are only used to help understand the method and core idea of the present invention. It should be noted that for those with professional skill in the field, without departing from the principle of the present invention, a plurality of improvements and modifications might also be made to the present invention, and these improvements and modifications also fall within the claims of the present invention.

What is claimed is:

1. A strain of Paenibacillus sp. for fermenting a wheat bran to synthesize an extracellular polysaccharide, wherein a preservation number of the strain is CGMCC NO.8333.

2. The strain of Paenibacillus sp. according to claim 1, wherein the strain is configured for fermenting the wheat bran to synthesize the extracellular polysaccharide.

3. The strain of Paenibacillus sp. according to claim 2, wherein a weight-average molecular weight of the extracellular polysaccharide synthesized by the strain is 300,800 daltons to 451,200 daltons.

4. The strain of Paenibacillus sp. according to claim 2, wherein the extracellular polysaccharide synthesized by the strain is an acidic heteropolysaccharide comprising glucuronic acid, glucose, and fucose in a molar ratio of (1.55 to 1.60): 1:(1.63 to 1.72).

5. The strain of Paenibacillus sp. according to claim 2, wherein a backbone of the extracellular polysaccharide synthesized by the strain comprises a 1,3-linked glucose residue, a 1,3-linked fucose residue, a 1,3,4-linked fucose residue,. and a 1,4-linked glucuronic acid residue, a branch point is located at a 0-4 position of the 1,3,4-linked fucose residue and a branch chain comprises terminally linked glucuronic acid residues.

6. The strain of Paenibacillus sp. according to claim 2, wherein the extracellular polysaccharide synthesized by the strain comprises a repeating unit shown in Formula I

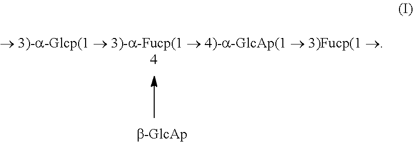

7. A method of use of the extracellular polysaccharide synthesized by the strain of Paenibacillus sp. according to claim 1 in fields of food and medicine.

8. A method of use of the extracellular polysaccharide synthesized by the strain of Paenibacillus sp. according to claim 1 in a preparation of immunomodulatory agents.

9. The method according to claim 7, wherein a concentration of the extracellular polysaccharide synthesized by the strain is lower than 100 μg/mL.

10. The method according to claim 7, wherein the strain is configured for fermenting the wheat bran to synthesize the extracellular polysaccharide.

11. The method according to claim 8, wherein the strain is configured for fermenting the wheat bran to synthesize the extracellular polysaccharide.

12. The method according to claim 10, wherein a concentration of the extracellular polysaccharide synthesized by the strain is lower than 100 μg/mL.

13. The method according to claim 8, wherein a concentration of the extracellular polysaccharide synthesized by the strain is lower than 100 μg/mL.

14. The method according to claim 11, wherein a concentration of the extracellular polysaccharide synthesized by the strain is lower than 100 μg/mL.
